(19) United States
(12) Patent Application Publication
(10) Pub. No.: US 2003/0216588 A1
(43) Pub. Date: Nov. 20, 2003

(54) Purification of 7alpha-hydroxydehydroepiandrosterone and derivatives thereof and solvates obtained therefrom

(75) Inventors: Marcel Fonteray, Breuil Le Sec (FR); Thi-My Ly-Carry, St Maur (FR); Jinzhu Xu, Paris (FR); Virginie Kompalitch, Paris (FR)

Correspondence Address: BURNS, DOANE, SWECKER & MATHIS, L.L.P., P.O. Box 1404, Alexandria, VA, 22313-1404, US

(73) Assignee: SOCIETE L'OREAL S.A., Paris (FR)

(21) Appl. No.: 10/394,176

(22) Filed: Mar. 24, 2003

Related U.S. Application Data
(60) Provisional application No. 60/367,713, filed on Mar. 28, 2002.

(30) Foreign Application Priority Data
Mar. 22, 2002 (FR) 02/03629

(51) Int. Cl. C07J 1/00

(52) U.S. Cl. 552/615

(57) ABSTRACT

Novel solvates of 7α-hydroxydehydroepiandrosterone or derivatives thereof complexed with an alcohol are obtained by judiciously purifying a mixture of the 7α-OH and 7β-OH isomers.

### CROSS-REFERENCE TO PRIORITY/PROVISIONAL APPLICATIONS

This application claims priority under 35 U.S.C. § 119 of FR-02/03629, filed Mar. 22, 2002, and of provisional application Serial No. 60/367,713, filed Mar. 28, 2002, both hereby expressly incorporated by reference. This application is also a continuation of said '713 provisional.

### BACKGROUND OF THE INVENTION

1. Technical Field of the Invention

The present invention relates to a novel process for the purification of 7α-hydroxydehydroepiandrosterone (7α-OH-DHEA) and of 7α-OH-DHEA derivatives, and to solvates obtained therefrom.

More specifically, the invention relates to a process for the purification of a compound of formula (Ia) or (Ib):

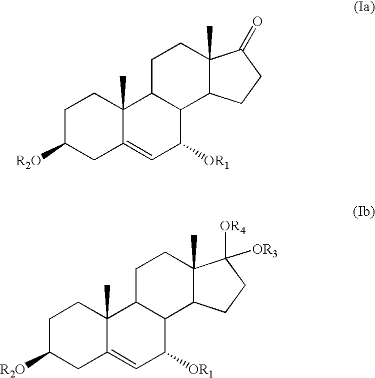

in which R₁ and R₂, which may be identical or different, are each H, alkyl, arylalkyl, alkylcarbonyl or arylcarbonyl, and —OR₃ and —OR₄, which may be identical or different, are each an alkoxy radical having 1 to 6 carbon atoms, such as methoxy, ethoxy or t-butoxy, or —OR₃ and —OR₄ may together form, with the carbon atom from what they depend, an optionally substituted —O—(CH₂)x-O— ring in which x ranges from 1 to 6 inclusive.

The compound of formula (Ia) wherein R₁ and R₂ are each H corresponds to 7α-hydroxydehydroepiandrosterone (7α-OH-DHEA).

Throughout the description of the present invention, by the term “alkyl” is intended a saturated and linear or branched hydrocarbon having from 1 to 6 carbon atoms.

By the term “alkoxy” is intended an RO— radical in which R is alkyl, for example methoxy, ethoxy or t-butoxy.

By the term “arylalkyl” is intended an alkyl radical substituted by an aryl moiety. And by the term “aryl” is intended an aromatic ring or double ring, such as, for example, phenyl, naphthyl or indanyl, optionally substituted by one to three substituents, such as alkyl, halogen or alkoxy.

By the term “alkylcarbonyl” is intended an R—CO— radical in which R is an alkyl radical; particularly exemplary is acetyl.

By the term “arylcarbonyl” is intended an Ar—CO— radical such that Ar is an aryl radical.

And the expression “optionally substituted” is intended that the —O—(CH₂)x-O— radical may or may not be substituted by one or more groups, such as halogen, alkyl, alkoxy or hydroxyl.

The present invention also relates to novel compounds formed by the derivative of formula (Ia) or (Ib) and an alcohol.

2. Description of the Prior Art

7α-OH-DHEA is a derivative well described in the scientific literature. The various synthetic processes described in the prior art generally result in a crude product in the form of a mixture composed of 7α-OH-DHEA, of the 7β-OH-DHEA isomer and of other by-products.

The production of the pure 7α stereoisomeric form is indicated, in particular, in FR-A1-2,771,105, WO-A1-92/03925 and FR-A1-2,793,491.

FR-A1-2,771,105 describes a process for the preparation of 7α-OH-DHEA from DHEA utilizing a fungus: Fusarium moniliforme. The product formed is purified by chromatography on a column of silica gel set up in ethyl acetate and eluted with ethyl acetate. This process is expensive and reproducible with difficulty in producing a large amount of compound.

WO-A1-92/03925 describes a process for the synthesis, chemically, starting from 3-O-acetyl-DHEA via the 3-O-acetyl-7-bromo-DHEA derivative. The desired product is purified by a process which consists in dissolving the crude product in acetone or in methanol, followed by precipitating with hexane. Not indicated is the yield obtained and the isomeric purity of the 7α-OH-DHEA is not specified. On repeating this process, the assignee hereof isolated a mixture of the 7α- and 7β-OH-DHEA isomers, the proportion of the 7β isomer of which was 30%.

FR-A1-2,793,491 describes a process which consists of a stereoselective synthesis by allylic oxidation of the 7-position of 3-O-acetyl-DHEA. The expected product is purified by chromatography on a column of silica gel, elution being carried out with a hexane/ethyl acetate mixture, in the initial proportions 50/50, gradually enriched in ethyl acetate up to 100% of ethyl acetate.

To summarize, it is very laborious, by the abovementioned processes, to obtain pure 7α-OH-DHEA or pure 7α-OH-DHEA derivatives. By the method of chromatography on silica gel, the elution times of the 7α and 7β isomers are very similar, rendering the purification operation difficult, expensive and difficult to carry out on an industrial scale. By the precipitation method, the 7β isomer is obtained as a mixture with the 7α isomer.

### SUMMARY OF THE INVENTION

The present invention thus features a process for the purification of the compounds of formula (Ia) or (Ib), in particular of 7α-OH-DHEA, which is simple, efficient and can be carried out on an industrial scale.

The process for the purification of the compounds of formula (Ia) or (Ib) according to the invention entails recrystallizing a crude reaction product, comprising a mixture of the 7α-OH isomer of formula (Ia) or (Ib) and of the corresponding 7β-OH isomer, and optionally of other compounds, from an alcoholic solvent or a mixture of solvents which comprises an alcoholic solvent and at least one solvent which is miscible with the said alcoholic solvent.

### DETAILED DESCRIPTION OF BEST MODE AND SPECIFIC/PREFERRED EMBODIMENTS OF THE INVENTION

More particularly according to the present invention, the solvent which is miscible with the said alcoholic solvent can be hydrocarbonaceous, for example heptane, petroleum ether and/or toluene.

By the term “7α-OH isomer” is intended, in the sense of the present invention, the compound of formula (Ia) or (Ib), in particular 7α-OH-DHEA or the 7α-OH-DHEA derivative. In addition, by the term “7β-OH isomer” is intended the β isomer corresponding to the 7α-OH isomer of formula (Ia) or (Ib).

The process of the invention advantageously permits obtaining an isomeric purity of 7α-OH isomer of greater than at least 90%, preferably of greater than 95%, more preferably of greater than 97%. It utilizes nontoxic solvents that are moderate in cost. Finally, it is simple to carry out.

More specifically, the present invention features a process for the purification of a 7α-OH derivative of formula (Ia) or (Ib) from a mixture comprising the 7α-OH isomer of formula (Ia) or (Ib) and the corresponding 7β-OH isomer, and optionally other compounds, said process comprising the stages:

(a) dissolving the mixture which comprises the said 7α-OH and 7β-OH isomers in an appropriate volume of an alcoholic solvent having one to ten carbon atoms, or a mixture of solvents comprising an alcoholic solvent having one to ten carbon atoms and at least one solvent which is miscible in the said alcoholic solvent,

(b) crystallizing the 7α-OH isomer, and

(c) filtering off and washing the 7α-OH isomer.

In one embodiment of the invention, the compounds of formula (Ia) with R₁ and R₂ denoting H (7α-OH-DHEA) or with R₁ representing H and R₂ representing acetyl (3-O-acetyl-7α-OH-DHEA) are preferred.

The alcoholic solvent preferably has 1 to 6, more preferably 2 to 4, carbon atoms. It is advantageously selected from among ethanol, n-propanol, tert-butanol, isopropanol, isobutanol and n-butanol, and mixtures thereof. Isopropanol is the preferred.

The mixture comprising the 7α-OH isomer and the 7β-OH isomer is dissolved in an appropriate volume of the said alcoholic solvent or of the said mixture of solvents, preferably 1 to 4 ml/g, more preferably from 1 to 2 ml/g, even more preferably approximately equal to 2 ml/g, of mixture of 7α-OH and 7β-OH isomers.

The recrystallization can be carried out by lowering the temperature or by simple evaporation of the solvent.

The crude reaction product is advantageously dissolved under hot conditions in the said alcoholic solvent or the said mixture of solvents, preferably at reflux. In this case, the solvate of the 7α-OH isomer advantageously crystallizes under cold conditions, in particular by cooling the solution to a temperature of less than or equal to approximately 10° C.

The alcoholic solvent or the mixture of solvents is optionally removed by drying under vacuum.

The process of the invention is advantageously carried out employing a crude reaction product which comprises a starting mixture of 7α-OH isomer of formula (Ia) or (Ib) and of corresponding 7β-OH isomer comprising at least 70% by weight of the 7α-OH isomer, it being understood that the process of the invention can be carried out employing a 7α/7β mixture in all proportions.

Thus, starting from a crude product with a 7α/7β ratio of 95/5, a purified product in a 7α/7β ratio of greater than 99/1 is generally obtained. Starting from a crude product with a ratio of 70/30, the ratio after purification is generally 98/2.

In another embodiment of the invention, the crude reaction product is obtained by one of the processes described in FR-A1-2,771,105, WO-A1-92/03925 and WO-A1-94/03176, which are incorporated in the present application by reference.

FR-A1-2,771,105 describes a process for the preparation of 7β-OH-DHEA in one stage from dehydroepiandrosterone (DHEA) using the fungus Fusarium moniliforme.

WO-A1-94/03176 and WO-A1-92/03925 describe a process for the preparation of 7α-OH-DHEA from 3-O-acetyl-DHEA by the chemical route in four stages. The first stage of this process makes it possible to obtain 3-O-acetyl-7-bromo-DHEA from 3-O-acetyl-DHEA by treatment with a brominating agent. In a second stage, the racemic mixture of isomers 7α-bromo are in the predominant form. After equilibration, the 3-O-acetyl-7-bromo-DHEA, treated with a mixture of glacial acetic acid and of silver acetate, permits obtaining 3-O-acetyl-7α-O-acetyl-DHEA. The 3-O-acetyl-7α-O-acetyl-DHEA is subsequently treated with sodium carbonate (Na₂CO₃) in a mixture of water and of methanol.

In yet another embodiment of the invention, the crude reaction product is obtained according to the process described in FR-2,820,745, also incorporated herein by reference.

This process essentially entails (i) carrying out an oxidation reaction in the allylic position of 3-O-acetyl-DHEA, then (ii) regioselectively and diastereoselectively reducing the 3-O-acetyl-7-oxo-DHEA using L-Selectride® as reducing agent, and in (iii) deprotecting the 7α-hydroxy-DHEA acetate by transesterification to obtain 7α-OH-DHEA.

Too, the present invention also features solvates, formed from 7α-OH-DHEA or a 7α-OH-DHEA derivative and an alcohol, said solvates having one of the following formulae:

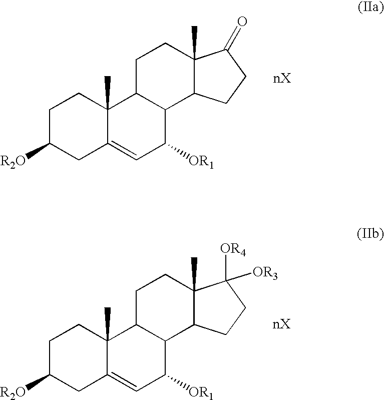

in which R₁ and R₂, which may be identical or different, are each H, alkyl, arylalkyl, alkylcarbonyl or arylcarbonyl; —OR₃ and —OR₄, which may be identical or different, are each an alkoxy radical having 1 to 6 carbon atoms, such as methoxy, ethoxy or t-butoxy, or —OR₃ and —OR₄ may together form, with the carbon atom from which they depend, an optionally substituted —O—(CH₂)x-O— ring wherein x ranges from 1 to 6, inclusive; n is a value of greater than 0 and less than 9; and X represents an alcohol having one to ten carbon atoms, preferably one to six, more preferably two to four, carbon atoms.

A particularly preferred solvate is that in which n has the value 1 and X is an aliphatic alcohol, such as isopropanol, tert-butanol, ethanol and/or n-butanol.

A particularly preferred solvate is that of formula (IIa).

A particularly preferred solvate is that in which R₁ and R₂ are each H (solvate of 7α-OH-DHEA) or R₁ represents H and R₂ represents acetyl (solvate of 3-O-acetyl-7α-OH-DHEA).

The physicochemical characteristics of the 7α-OH-DHEA solvate such that n has the value 1 and X represents isopropanol are reported in the Example 1 below.

The value of n can be equal to 0.5, 1, 1.5, 2 or 2.5, for example, as molecules of alcoholic solvent can form bonds with two different molecules of 7α-OH isomer.

In order to further illustrate the present invention and the advantages thereof, the following specific examples are given, it being understood that same are intended only as illustrative and in nowise limitative.

### EXAMPLE 1:

Preparation of the 7α-OH-DHEA-isopropanol solvate:

The process described in Example 1 of FR-2,820,745 was repeated, starting with 30 g of 3-O-acetyl-7-keto-DHEA, to synthesize the mixture of 7α-OH-DHEA and 7p-OH-DHEA isomers. The crude product thus obtained was dissolved in 60 ml of isopropanol (i.e., 2 ml/g) at reflux. The solution was subsequently cooled to +10° C. The crystals were filtered off and washed with a cold heptane/isopropanol (1/1) mixture to give 14.6 g of a white powder.

Characterizations:

¹H NMR (d₆-DMSO, 200 MHZ) δ (ppm): conforms to the expected product.

7α/7β ratio by HPLC: >99%.

DSC profile: peak for departure of solvent at 116.0° C. and then melting peak at 170.6° C., corresponding to the 7α-OH-DHEA.isopropanol solvate.

Quantitative determination of isopropanol by gas chromatography: ˜16%.

Elemental analysis for C₁₉H₂₈O₃.C₃H₈O:

% Theory: C: 72.48 H : 9.95 O : 17.56

% Found: C: 72.88 H : 9.88 O : 16.76

### EXAMPLE 2:

The procedure of Example 1 was repeated using other alcoholic solvents. The results obtained are reported in the following Table:

TABLE

| | AMOUNT OF | |
|---|---|---|
| | RECRYSTALLIZATION | 7α/7β RATIO BY |
| SOLVENT | SOLVENT | HPLC |
| ethanol | 1 ml/g | 99.2/0.8 |
| n-butanol | 1 ml/g | 98.4/1.6 |
| tert-butanol | 2.3 ml/g | 99.5/0.5 |

Each patent, patent application and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A solvate of 7α-hydroxydehydroepiandrosterone (7α-OH-DHEA) or of a 7α-OH-DHEA derivative with an alcohol, said solvate having one of the following structural formulae: 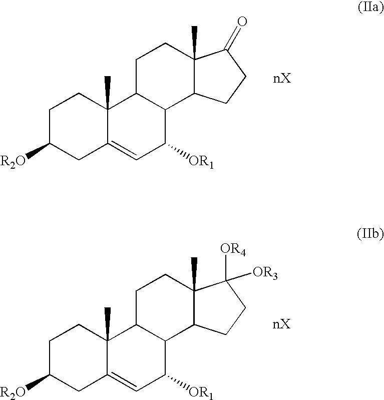
in which R₁ and R₂, which may be identical or different, are each H, alkyl, arylalkyl, alkylcarbonyl or arylcarbonyl; —OR₃ and —OR₄, which may be identical or different, are each an alkoxy radical having 1 to 6 carbon atoms, or —OR₃ and —OR₄ may together form, with the carbon atom from which they depend, an optionally substituted —O—(CH₂)x-O— ring wherein x ranges from 1 to 6, inclusive; n is a value of greater than 0 and less than 9; and X represents an alcohol having one to ten carbon atoms.

2. The solvate as defined by claim 1, wherein formulae (IIa) or (IIb), —OR₃ and —OR₄ are each methoxy, ethoxy or t-butoxy.

3. The solvate as defined by claim 1, wherein formulae (IIa) or (IIb), X is an alcohol having one to six carbon atoms.

4. The solvate as defined by claim 1, wherein formulae (IIa) or (IIb), X is an alcohol having two to four carbon atoms.

5. The solvate as defined by claim 1, having formula (IIa), wherein either R₁ and R₂ is H or R₁ is H and R₂ is acetyl.

6. The solvate as defined by claim 1, wherein X is an aliphatic alcohol.

7. The solvate as defined by claim 6, wherein X is isopropanol, tert-butanol, ethanol and/or n-butanol.

8. The solvate as defined by claim 1, wherein formulae (IIa) or (IIb), X is isopropanol and n has the value 1.

9. A process for the purification of a compound of formula (Ia) or (Ib): 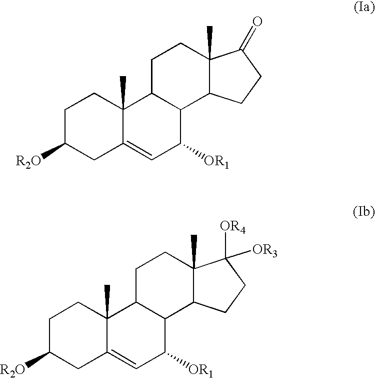
in which R₁ and R₂, which may be identical or different, are each H, alkyl, arylalkyl, alkylcarbonyl or arylcarbonyl; and —OR₃ and —OR₄, which may be identical or different, are each an alkoxy radical having 1 to 6 carbon atoms, or —OR₃ and —OR₄ may together form, with the carbon atom from which they depend, an optionally substituted —O—(CH₂)x-O— ring in which x ranges from 1 to 6, inclusive, from a mixture which comprises the 7α-OH isomer of formula (Ia) or (Ib) and the corresponding 7β-OH isomer, said process comprising:
(a) dissolving the mixture which comprises the said 7α-OH and 7β-OH isomers in an appropriate volume of an alcoholic solvent having one to ten carbon atoms, or a mixture of solvents comprising an alcoholic solvent having one to ten carbon atoms and at least one solvent which is miscible in the said alcoholic solvent,
(b) crystallizing the 7α-OH isomer, and
(c) filtering off and washing the 7α-OH isomer.

10. The process as defined by claim 9, the compound purified having formula (Ia) wherein R₁ and R₂ are each H (7α-OH-DHEA) or R₁ is H and R₂ is acetyl.

11. The process as defined by claim 9, said alcoholic solvent having 1 to 6 carbon atoms.

12. The process as defined by claim 11, said alcoholic solvent comprising isopropanol, ethanol, n-propanol, tert-butanol, isobutanol, n-butanol or mixtures thereof.

13. The process as defined by claim 9, wherein the volume of alcoholic solvent or mixture of solvents ranges from 1 and 4 ml/g, of mixture of 7α-OH isomer and of 7β-OH isomer.

14. The process as defined by claim 9, wherein the mixture of 7α-OH isomer and of 7β-OH isomer is dissolved under hot or reflux conditions in the alcoholic solvent or mixture of solvents.

15. The process as defined by claim 9, wherein the starting mixture of 7α-OH isomer and of 7β-OH isomer comprises at least 70% by weight of 7α-OH isomer.

16. The process as defined by claim 13, wherein the volume of alcoholic solvent or mixture of solvents ranges from 1 to 2 ml/g, of mixture of 7α-OH isomer and of 7β-OH isomer.

17. The process as defined by claim 16, wherein the volume of alcoholic solvent or mixture of solvents is approximately 2 ml/g, of mixture of 7α-OH isomer and of 7β-OH isomer.
